(19) United States
(12) Patent Application Publication
(10) Pub. No.: US 2013/0104573 A1
(43) Pub. Date: May 2, 2013

(54) USE OF COMPOSITIONS COMPRISING 1,1,1,2,3-PENTAFLUOROPROPANE AND OPTIONALLY Z-1,1,1,4,4,4-HEXAFLUORO-2-BUTENE IN CHILLERS

(71) Applicant: E I Du Pont De Nemours and Company, Wilmington, DE (US)

(72) Inventor: KONSTANTINOS KONTOMARIS, Wilmington, DE (US)

(73) Assignee: E I DU PONT DE NEMOURS AND COMPANY, Wilmington, DE (US)

(21) Appl. No.: 13/666,360

(22) Filed: Nov. 1, 2012

Related U.S. Application Data
(60) Provisional application No. 61/554,768, filed on Nov. 2, 2011.

(51) Int. Cl. C07C 19/08; F25B 39/02; F25B 1/00; C09K 5/04

(52) U.S. Cl. 62/56; 252/68; 570/134; 62/498

(57) ABSTRACT

A method is provided for producing cooling in a chiller having an evaporator wherein a refrigerant composition is evaporated to cool a heat transfer medium. The method comprises evaporating a refrigerant composition comprising HFC-245eb and optionally Z—HFO-1336mzz in the evaporator. Additionally, a composition is provided comprising: (1) a refrigerant composition consisting essentially of HFC-245eb and Z—HFO-1336mzz; (2) a lubricant suitable for use in a chiller; wherein the Z—HFO-1336mzz in the refrigerant composition is at least about 41 weight percent. Also, a chiller apparatus is provided comprising an evaporator, a compressor, a condenser and a pressure reduction device, all of which are in fluid communication in the order listed and through which a refrigerant flows from one component to the next in a repeating cycle.

### CROSS REFERENCE(S) TO RELATED APPLICATION(S)

This application claims the priority benefit of U.S. Provisional Patent Application No. 61/554,768, filed Nov. 2, 2011.

### FIELD OF THE INVENTION

This invention relates to methods and systems having for producing cooling in numerous applications, and in particular, in chillers.

### BACKGROUND OF THE INVENTION

The compositions of the present invention are part of a continued search for the next generation of low global warming potential materials. Such materials must have low environmental impact, as measured by low global warming potential and zero ozone depletion potential. New chiller working fluids are needed.

### SUMMARY OF THE INVENTION

The present invention involves compositions comprising 1,1,1,2,3-pentafluoropropane (HFC-245eb) and optionally Z-1,1,1,4,4,4-hexafluoro-2-butene (Z—HFO-1336mzz).

Embodiments of the present invention involve the compound HFC-245eb, either alone or in combination with one or more other compounds as described in detail herein below.

In accordance with the present invention a method is provided for producing cooling in a chiller having an evaporator wherein a refrigerant composition is evaporated to cool a heat transfer medium and the cooled heat transfer medium is transported out of the evaporator to a body to be cooled, comprising: evaporating a refrigerant composition comprising HFC-245eb and optionally Z—HFO-1336mzz in the evaporator.

Also in accordance with the present invention a composition is provided comprising: (1) a refrigerant composition consisting essentially of HFC-245eb and Z—HFO-1336mzz; (2) a lubricant suitable for use in a chiller; wherein the Z—HFO-1336mzz in the refrigerant composition is at least about 41 weight percent.

Also in accordance with the present invention a chiller apparatus is provided containing a refrigerant composition comprising HFC-245eb and optionally Z—HFO-1336mzz. The chiller apparatus may comprise (a) an evaporator through which a refrigerant flows and is evaporated; (b) a compressor in fluid communication with the evaporator that compresses the evaporated refrigerant to a higher pressure; (c) a condenser in fluid communication with the compressor through which the high pressure refrigerant vapor flows and is condensed; and (d) a pressure reduction device in fluid communication with the condenser wherein the pressure of the condensed refrigerant is reduced and said pressure reduction device further being in fluid communication with the evaporator such that the refrigerant then repeats flow through components (a), (b), (c) and (d) in a repeating cycle; wherein said refrigerant comprises HFC-245eb and optionally Z—HFO-1336mzz.

### BRIEF DESCRIPTION OF THE DRAWINGS

FIG. 1 is a schematic diagram of one embodiment of a centrifugal chiller having a flooded evaporator, which utilizes a composition comprising HFC-245eb and optionally Z—HFO-1336mzz.

FIG. 2 is a schematic diagram of one embodiment of a centrifugal chiller having a direct expansion evaporator, which utilizes a composition comprising HFC-245eb and optionally Z—HFO-1336mzz.

### DETAILED DESCRIPTION OF PREFERRED EMBODIMENTS

Before addressing details of embodiments described below, some terms are defined or clarified.

Global warming potential (GWP) is an index for estimating relative global warming contribution due to atmospheric emission of a kilogram of a particular greenhouse gas compared to emission of a kilogram of carbon dioxide. GWP can be calculated for different time horizons showing the effect of atmospheric lifetime for a given gas. The GWP for the 100 year time horizon is commonly the value referenced.

Ozone depletion potential (ODP) is defined in “The Scientific Assessment of Ozone Depletion, 2002, A report of the World Meteorological Association's Global Ozone Research and Monitoring Project,” section 1.4.4, pages 1.28 to 1.31 (see first paragraph of this section). ODP represents the extent of ozone depletion in the stratosphere expected from a compound on a mass-for-mass basis relative to fluorotrichloromethane (CFC-11).

Refrigeration capacity (sometimes referred to as cooling capacity) is a term to define the change in enthalpy of a refrigerant composition in an evaporator per unit mass of refrigerant composition circulated. Volumetric cooling capacity refers to the amount of heat removed by the refrigerant composition in the evaporator per unit volume of refrigerant composition vapor exiting the evaporator. The refrigeration capacity is a measure of the ability of a refrigerant composition or heat transfer composition to produce cooling. Cooling rate refers to the heat removed by the refrigerant composition in the evaporator per unit time.

Coefficient of performance (COP) is the amount of heat removed in an evaporator divided by the energy required to operate a compressor. The higher the COP, the higher the energy efficiency. COP is directly related to the energy efficiency ratio (EER), that is, the efficiency rating for refrigeration or air conditioning equipment at a specific set of internal and external temperatures.

As used herein, a heat transfer medium comprises a composition used to carry heat from a body to be cooled to the chiller evaporator or from the chiller condenser to a cooling tower or other configuration where heat can be rejected to the ambient.

As used herein, a refrigerant composition is a composition which may be a single compound or comprise a mixture of compounds that functions to transfer heat in a cycle wherein the composition undergoes a phase change from a liquid to a gas and back to a liquid in a repeating cycle.

Subcooling is the reduction of the temperature of a liquid below that liquid's saturation point for a given pressure. The saturation point is the temperature at which a vapor composition is completely condensed to a liquid (also referred to as the bubble point). But subcooling continues to cool the liquid to a lower temperature liquid at the given pressure. By cooling a liquid below the saturation temperature, the net refrigeration capacity can be increased. Subcooling thereby improves refrigeration capacity and energy efficiency of a system. Subcool amount is the amount of cooling below the saturation temperature (in degrees) or how far below its saturation temperature a liquid composition is cooled.

Superheat is a term that defines how far above the saturation vapor temperature of a vapor composition a vapor composition is heated. Saturation vapor temperature is the temperature at which, if a vapor composition is cooled, the first drop of liquid is formed, also referred to as the “dew point”.

Temperature glide (sometimes referred to simply as “glide”) is the absolute value of the difference between the starting and ending temperatures of a phase-change process by a refrigerant composition within a component of a refrigerant system, exclusive of any subcooling or superheating. This term may be used to describe condensation or evaporation of a near azeotrope or non-azeotropic composition. Average glide refers to the average of the glide in the evaporator and the glide in the condenser of a specific chiller system operating under a given set of conditions.

An azeotropic composition is a mixture of two or more different components which, when in liquid form under a given pressure, will boil at a substantially constant temperature, which temperature may be higher or lower than the boiling temperatures of the individual components, and which will provide a vapor composition essentially identical to the overall liquid composition undergoing boiling. (see, e.g., M. F. Doherty and M. F. Malone, Conceptual Design of Distillation Systems, McGraw-Hill (New York), 2001, 185-186, 351-359).

Accordingly, the essential features of an azeotropic composition are that at a given pressure, the boiling point of the liquid composition is fixed and that the composition of the vapor above the boiling composition is essentially that of the overall boiling liquid composition (i.e., no fractionation of the components of the liquid composition takes place). It is also recognized in the art that both the boiling point and the weight percentages of each component of the azeotropic composition may change when the azeotropic composition is subjected to boiling at different pressures. Thus, an azeotropic composition may be defined in terms of the unique relationship that exists among the components or in terms of the compositional ranges of the components or in terms of exact weight percentages of each component of the composition characterized by a fixed boiling point at a specified pressure.

For the purpose of this invention, an azeotrope-like composition means a composition that behaves substantially like an azeotropic composition (i.e., has constant boiling characteristics or a tendency not to fractionate upon boiling or evaporation). Hence, during boiling or evaporation, the vapor and liquid compositions, if they change at all, change only to a minimal or negligible extent. This is to be contrasted with non-azeotrope-like compositions in which during boiling or evaporation, the vapor and liquid compositions change to a substantial degree.

As used herein, the terms “comprises,” “comprising,” “includes,” “including,” “has,” “having” or any other variation thereof, are intended to cover a non-exclusive inclusion. For example, a composition, process, method, article, or apparatus that comprises a list of elements is not necessarily limited to only those elements but may include other elements not expressly listed or inherent to such composition, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

The transitional phrase “consisting of” excludes any element, step, or ingredient not specified. If in the claim such would close the claim to the inclusion of materials other than those recited except for impurities ordinarily associated therewith. When the phrase “consists of” appears in a clause of the body of a claim, rather than immediately following the preamble, it limits only the element set forth in that clause; other elements are not excluded from the claim as a whole.

The transitional phrase “consisting essentially of” is used to define a composition, method or apparatus that includes materials, steps, features, components, or elements, in addition to those literally disclosed provided that these additional included materials, steps, features, components, or elements do materially affect the basic and novel characteristic(s) of the claimed invention. The term ‘consisting essentially of’ occupies a middle ground between “comprising” and ‘consisting of’.

Where applicants have defined an invention or a portion thereof with an open-ended term such as “comprising,” it should be readily understood that (unless otherwise stated) the description should be interpreted to also describe such an invention using the terms “consisting essentially of” or “consisting of.”

Also, use of “a” or “an” are employed to describe elements and components described herein. This is done merely for convenience and to give a general sense of the scope of the invention. This description should be read to include one or at least one and the singular also includes the plural unless it is obvious that it is meant otherwise.

Unless otherwise defined, all technical and scientific terms used herein have the same meaning as commonly understood by one of ordinary skill in the art to which this invention belongs. Although methods and materials similar or equivalent to those described herein can be used in the practice or testing of embodiments of the present invention, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

HFC-245eb, or 1,1,1,2,3-pentafluoropropane (CF₃CHFCH₂F), can be prepared by the hydrogenation of 1,1,1,2,3-pentafluoro-2,3,3-trichloropropane (CF₃CClFCCl₂F or CFC-215bb) over a palladium on carbon catalyst as disclosed in U.S. Patent Publication No. 2009-0264690 A1, incorporated herein in its entirety, or by hydrogenation of 1,2,3,3,3-pentafluoropropene (CF₃CF═CFH or HFO-1225ye) as disclosed in U.S. Pat. No. 5,396,000, incorporated herein by reference.

Z-1,1,1,4,4,4-hexafluoro-2-butene (also known as Z—HFO-1336mzz or cis-HFO-1336mzz and having the structure cis-CF₃CH═CHCF₃), may be made by methods known in the art, such as by hydrodechlorination of 2,3-dichloro-1,1,1,4,4,4-hexafluoro-2-butene, as described in U.S. Patent Application Publication No. US 2009/0012335A1, incorporated herein by reference.

### Chiller Methods

A method is provided for producing cooling in a chiller having an evaporator wherein a refrigerant composition is evaporated to cool a heat transfer medium and the cooled heat transfer medium is transported out of the evaporator to a body to be cooled. The method comprises evaporating a refrigerant composition comprising HFC-245eb and optionally Z—HFO-1336mzz in the evaporator. In one embodiment the method comprises (a) passing a heat transfer medium through an evaporator; (b) evaporating a liquid refrigerant composition comprising HFC-245eb and optionally Z—HFO-1336mzz in the evaporator thereby producing a vapor refrigerant composition; and (b) compressing the vapor refrigerant composition in a compressor. The compressor may be a positive displacement compressor or a centrifugal compressor. Positive displacement compressors include reciprocating, screw, or scroll compressors. Of note are methods for producing cooling that use centrifugal compressors. The method for producing cooling typically provides cooling to an external location to which the cooled heat transfer medium passes from the evaporator to a body to be cooled.

Neat HFC-245eb has been found to provide good cooling performance in chillers. Additionally, neat HMF-245eb has been found to match the performance for CFC-11 (fluorotrichloromethane) in chillers. And neat HMF-245eb has been found to be an improvement over use of HCFC-123 (2,2-dichloro-1,1,1-trifluoroethane) in chillers. Of note are methods for producing cooling wherein the refrigerant composition evaporated consists essentially of HFC-245eb.

While neat HFC-245eb meets the need for a chiller refrigerant composition, it can be improved by the addition of a component such as Z—HFO-1336mzz. Addition of Z—HFO-1336mzz to HFC-245eb gives the advantage of reducing the pressure and of reducing the GWP. Of particular utility in the method for producing cooling are those embodiments wherein the refrigerant composition consists essentially of a composition comprising HFC-245eb and optionally Z—HFO-1336mzz. Also of particular utility are those embodiments wherein the refrigerant composition is azeotropic or azeotrope-like. Because azeotropic and azeotrope-like compositions do not fractionate to any large degree, they function in a system with low temperature glide in the evaporator of the chiller.

Of note are compositions that provide less than 1° C. average temperature glide comprising less than or equal to about 57 weight percent Z—HFO-1336mzz and greater than or equal to about 43 weight percent HFC-245eb; or comprising greater than or equal to about 82 weight percent Z—HFO-1336mzz and less than or equal to about 18 weight percent HFC-245eb. Of particular note are compositions that provide less than 0.5° C. average temperature glide comprising less than or equal to about 35 weight percent Z—HFO-1336mzz and greater than or equal to about 65 weight percent HFC-245eb; or comprising greater than or equal to about 92 weight percent Z—HFO-1336mzz and less than or equal to about 8 weight percent HFC-245eb.

Also of note are methods for producing cooling wherein the refrigerant composition evaporated consists essentially of HFC-245eb and Z—HFO-1336mzz; and wherein the Z—HFO-1336mzz in the refrigerant composition is at least about 1 weight percent. Of particular note are methods of producing cooling wherein the refrigerant composition evaporated consists essentially of from about 99 weight percent to about 43 weight percent HFC-245eb and form about 1 weight percent to about 57 weight percent Z—HFO-1336mzz. Also of particular note are methods of producing cooling wherein the refrigerant composition evaporated consists essentially of from about 1 weight percent to about 18 weight percent HFC-245eb and form about 82 weight percent to about 99 weight percent Z—HFO-1336mzz.

Certain refrigerant compositions of this invention consists essentially of from about 99 weight percent to about 43 weight percent HFC-245eb and form about 1 weight percent to about 57 weight percent Z—HFO-1336mzz. In one embodiment, non-flammable compositions are desirable for use in chillers. Of note are non-flammable compositions of this invention comprising at least 41 weight percent Z—HFO-1336mzz and no more than 59 weight percent HFC-245eb.

Of note are methods for producing cooling wherein the chiller evaporator is suitable for use with HCFC-123 and wherein the refrigerant composition consists essentially of from about 1 weight percent to about 59 weight percent HFC-245eb and from about 41 weight percent to about 99 weight percent Z—HFO-1336mzz.

Also of note are methods for producing cooling wherein said chiller is suitable for use with CFC-11 and wherein the refrigerant composition consists essentially of from about 1 weight percent to about 59 weight percent HFC-245eb and from 41 weight percent to about 99 weight percent Z—HFO-1336mzz.

Additionally, in another embodiment, chillers operated with Z—HFO-1336mzz/HFC-245eb blends containing about 71 weight percent or more Z—HFO-1336mzz will have vapor pressures below the threshold necessitating compliance with provisions of the ASME Boiler and Pressure Vessel Code. Such compositions are desirable for use in chillers.

Of note are methods for producing cooling wherein the chiller is suitable for use with HCFC-123 and wherein the refrigerant composition consists essentially of from about 1 weight percent to about 29 weight percent HFC-245eb and from about 71 weight percent to about 99 weight percent Z—HFO-1336mzz. Of particular note are compositions where the refrigerant composition consists essentially of from about 71 to about 80 weight percent Z—HFO-1336mzz and from about 29 to 20 weight percent HFC-245eb.

Further, in another embodiment, low GWP compositions are desirable. Of note are compositions comprising at least 49.5 weight percent Z—HFO-1336mzz and no more than 50.5 weight percent HFC-245eb, which have GWP less than 150.

In one embodiment, a body to be cooled may be any space, object or fluid that may be cooled. In one embodiment, a body to be cooled may be a room, building, passenger compartment of an automobile, refrigerator, freezer, or supermarket or convenience store display case. Alternatively, in another embodiment, a body to be cooled may be a heat transfer medium or heat transfer fluid.

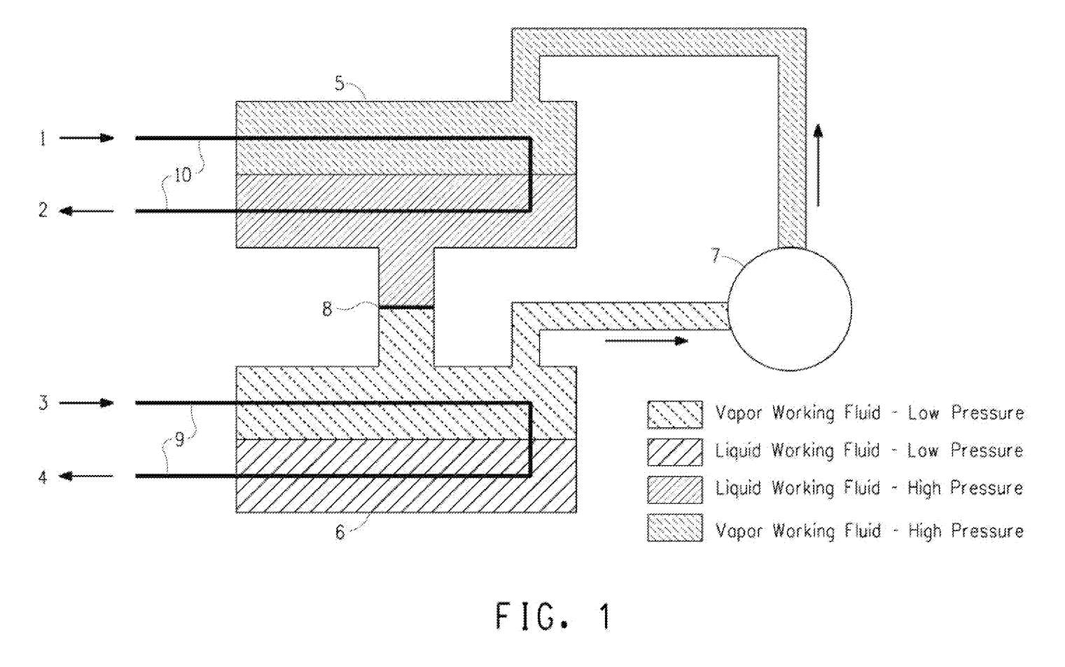

In one embodiment, the method for producing cooling comprises producing cooling in a flooded evaporator chiller as described above with respect to FIG. 1, as described in more detail hereinbelow. In this method, the refrigerant composition comprising HFC-245eb and optionally Z—HFO-1336mzz is evaporated to form refrigerant composition vapor in the vicinity of a first heat transfer medium. The heat transfer medium is a warm liquid, such as water, which is transported into the evaporator via a pipe from a cooling system. The warm liquid is cooled and is passed to a body to be cooled, such as a building. The refrigerant composition vapor is then condensed in the vicinity of a second heat transfer medium, which is a chilled liquid which is brought in from, for instance, a cooling tower. The second heat transfer medium cools the refrigerant composition vapor such that it is condensed to form a liquid refrigerant composition. In this method, a flooded evaporator chiller may also be used to cool hotels, office buildings, hospitals and universities.

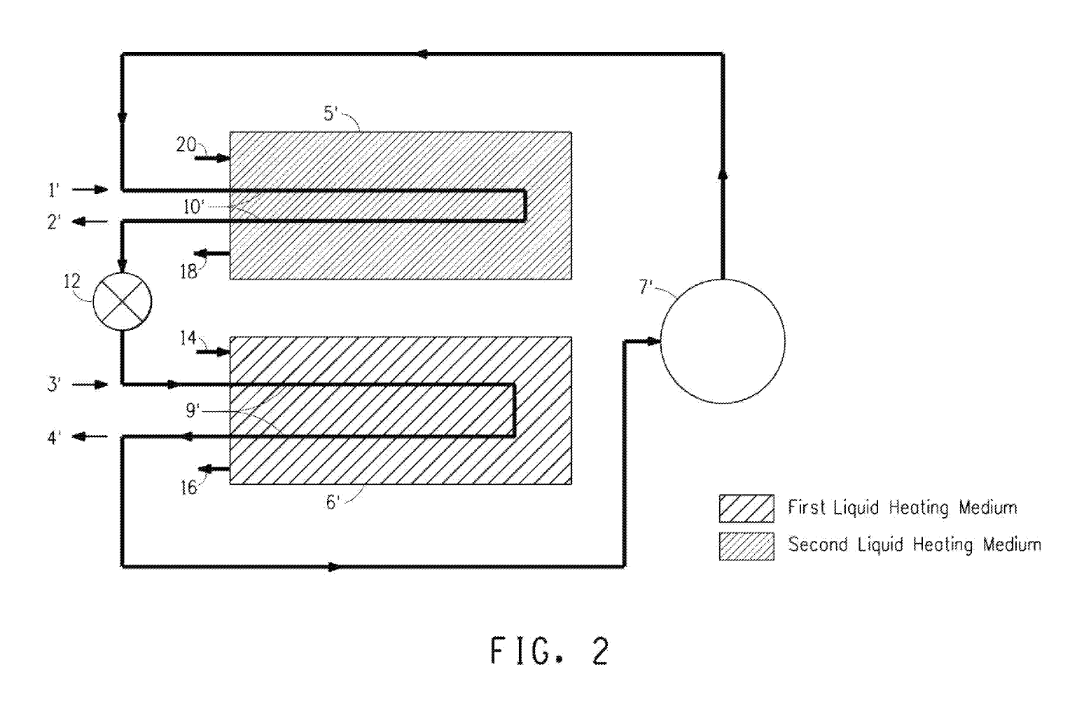

In another embodiment, the method for producing cooling comprises producing cooling in a direct expansion chiller as described above with respect to FIG. 2, as described in more detail below. In this method, the refrigerant composition comprising HFC-245eb and optionally Z—HFO-1336mzz is passed through an evaporator and evaporates to produce a refrigerant composition vapor. A first liquid heat transfer medium is cooled by the evaporating refrigerant composition. The first liquid heat transfer medium is passed out of the evaporator to a body to be cooled. In this method, the direct expansion chiller may also be used to cool hotels, office buildings, hospitals, universities, as well as naval submarines or naval surface vessels.

In either method for producing cooling in either a flooded evaporator chiller or in direct expansion chiller, the chiller may include a centrifugal compressor.

Refrigerant compositions and heat transfer fluids that are in need of replacement, based upon their GWP values published by the Intergovernmental Panel on Climate Change (IPCC), include but are not limited to HCFC-123. Therefore, in accordance with the present invention, there is provided a method for replacing HCFC-123 in a chiller. The method for replacing a refrigerant composition in a chiller designed for using HCFC-123 as refrigerant composition comprises charging said chiller with a composition comprising a refrigerant composition consisting essentially of HFC-245eb and optionally Z—HFO-1336mzz.

In this method of replacing HCFC-123, the refrigerant composition consists essentially of a HFC-245eb and optionally Z—HFO-1336mzz and is useful in centrifugal chillers that may have been originally designed and manufactured to operate with HCFC-123.

In replacing HCFC-123 with the refrigerant compositions as disclosed herein consisting essentially of HFC-245eb and optionally Z—HFO-1336mzz in existing equipment, additional advantages may be realized by making adjustments to equipment or operating conditions or both. For example, impeller diameter and impeller speed may be adjusted in a centrifugal chiller where a composition is being used as a replacement working fluid.

Alternatively, in the methods of replacing HCFC-123, the refrigerant composition consists essentially of HFC-245eb and optionally Z—HFO-1336mzz may be useful in new equipment, such as a new chiller comprising a flooded evaporator or a new chiller comprising a direct expansion evaporator.

### Chiller Apparatus

In one embodiment is provided a chiller apparatus containing a composition comprising a refrigerant composition comprising HFC-245eb and optionally Z—HFO-1336mzz. A chiller apparatus can be of various types including centrifugal apparatus and positive displacement apparatus. Chiller apparatus typically includes an evaporator, compressor, condenser and a pressure reduction device, such as a valve. Of note is a chiller apparatus comprising a refrigerant composition consisting essentially of HFC-245eb and optionally Z—HFO-1336mzz.

In one embodiment, the chiller apparatus comprises an evaporator, a compressor, a condenser and a pressure reduction device, all of which are in fluid communication in the order listed and through which a refrigerant flows from one component to the next in a repeating cycle.

In one embodiment the chiller apparatus comprises (a) an evaporator through which a refrigerant flows and is evaporated; (b) a compressor in fluid communication with the evaporator that compresses the evaporated refrigerant to a higher pressure; (c) a condenser in fluid communication with the compressor through which the high pressure refrigerant vapor flows and is condensed; and (d) a pressure reduction device in fluid communication with the condenser wherein the pressure of the condensed refrigerant is reduced and said pressure reduction device further being in fluid communication with the evaporator such that the refrigerant then repeats flow through components (a), (b), (c) and (d) in a repeating cycle.

Of particular utility in the chiller apparatus are those embodiments wherein the refrigerant composition consists essentially of a composition comprising HFC-245eb and optionally Z—HFO-1336mzz. Also of particular utility are those embodiments wherein the refrigerant composition is azeotropic or azeotrope-like. Because azeotropic and azeotrope-like compositions do not fractionate to any large degree, they function in a system with zero or low temperature glide in the evaporator and condenser of the chiller.

Of note are compositions that provide less than 1° C. average temperature glide comprising less than or equal to about 57 weight percent Z—HFO-1336mzz and greater than or equal to about 43 weight percent HFC-245eb; or comprising greater than or equal to about 82 weight percent Z—HFO-1336mzz and less than or equal to about 18 weight percent HFC-245eb. Of particular note are compositions that provide less than 0.5° C. average temperature glide comprising less than or equal to about 35 weight percent Z—HFO-1336mzz and greater than or equal to about 65 weight percent HFC-245eb; or comprising greater than or equal to about 92 weight percent Z—HFO-1336mzz and less than or equal to about 8 weight percent HFC-245eb.

In another embodiment, non-flammable compositions are desirable for use in chillers. Of note are non-flammable compositions comprising at least 41 weight percent Z—HFO-1336mzz and no more than 59 weight percent HFC-245eb.

Additionally, in another embodiment, chillers operated with Z—HFO-1336mzz/HFC-245eb blends containing about 71 weight percent or more of Z—HFO-1336mzz will have vapor pressures below the threshold necessitating compliance with provisions of the ASME Boiler and Pressure Vessel Code. Such compositions are desirable for use in chillers. Of note are compositions where the refrigerant composition consists essentially of from about 71 to about 80 weight percent Z—HFO-1336mzz and from about 29 to 20 weight percent HFC-245eb.

Further, in another embodiment, low GWP compositions are desirable. Of note are compositions comprising at least 49.5 weight percent Z—HFO-1336mzz and no more than 50.5 weight percent HFC-245eb, which have GWP less than 150.

A chiller is a type of air conditioning/refrigeration apparatus. The present disclosure is directed to a vapor compression chiller. Vapor compression chillers include components, such as a compressor, a condenser, an expansion device and an evaporator. Such vapor compression chillers may be either flooded evaporator chillers, one embodiment of which is shown in FIG. 1, or direct expansion chillers, one embodiment of which is shown in FIG. 2. Both a flooded evaporator chiller and a direct expansion chiller may be air-cooled or water-cooled. In the embodiment where chillers are water cooled, such chillers are generally associated with cooling towers for heat rejection from the system. In the embodiment where chillers are air-cooled, the chillers are equipped with refrigerant-to-air finned-tube condenser coils and fans to reject heat from the system. Air-cooled chiller systems are generally less costly than equivalent-capacity water-cooled chiller systems including cooling tower and water pump. However, water-cooled systems can be more efficient under many operating conditions due to lower condensing temperatures.

Chillers, including both flooded evaporator and direct expansion chillers, may be coupled with an air handling and distribution system to provide comfort air conditioning (cooling and dehumidifying the air) to large commercial buildings, including hotels, office buildings, hospitals, universities and the like. In another embodiment, chillers, most likely air-cooled direct expansion chillers, have found additional utility in naval submarines and surface vessels.

To illustrate how chillers operate, reference is made to the Figures. A water-cooled, flooded evaporator chiller is illustrated in FIG. 1. In this chiller a first heat transfer medium, which is a warm liquid comprising water, and, in some embodiments, additives, such as a glycol (e.g., ethylene glycol or propylene glycol), enters the chiller from a cooling system, such as a building cooling system. The first heat transfer medium is shown entering the chiller at arrow 3, through coil or tube bundle 9, in evaporator 6, which has an inlet and an outlet. The warm first heat transfer medium is delivered to evaporator 6, where it is cooled by liquid refrigerant composition, which is shown in the lower portion of evaporator 6 as liquid working fluid—low pressure. The liquid refrigerant composition evaporates at a temperature lower than the temperature of the warm first heat transfer medium which flows through coil 9. The cooled first heat transfer medium re-circulates back to the building cooling system, as shown by arrow 4, via a return portion of coil 9. The liquid refrigerant composition, shown in the lower portion of evaporator 6 as liquid working fluid—low pressure, vaporizes to form vapor working fluid—low pressure in upper portion of evaporator 6, and is drawn into compressor 7, which increases the pressure and temperature of the refrigerant composition vapor (vapor working fluid). Compressor 7 compresses this vapor so that it may be condensed in condenser 5 at a higher pressure and temperature than the pressure and temperature of the refrigerant composition vapor when from evaporator 6. A second heat transfer medium, which is a liquid in the case of a water-cooled chiller, enters condenser 5 via coil or tube bundle 10 in condenser 5 from a cooling tower at arrow 1. The second heat transfer medium is warmed in the process and returned via a return loop of coil 10 and arrow 2 to a cooling tower or to the environment. This second heat transfer medium cools the vapor in condenser 5 and causes the vapor to condense to liquid refrigerant composition, so that there is liquid refrigerant composition (liquid working fluid—high pressure) in the lower portion of condenser 5. The condensed liquid refrigerant composition in condenser 5 flows back to evaporator 6 through expansion device 8, which may be an orifice, capillary tube or expansion valve. Expansion device 8 reduces the pressure of the liquid refrigerant composition, and converts the liquid refrigerant composition partially to vapor, that is to say that the liquid refrigerant composition flashes as pressure drops between condenser 5 and evaporator 6. Flashing cools the refrigerant composition, i.e., both the liquid refrigerant composition and the refrigerant composition vapor to the saturation temperature at evaporator pressure, so that both liquid refrigerant composition and refrigerant composition vapor are present in evaporator 6.

It should be noted that for a single component refrigerant composition, the composition of the vapor refrigerant composition in the evaporator is the same as the composition of the liquid refrigerant composition in the evaporator. In this case, evaporation will occur at a constant temperature. However, if a refrigerant composition which is a blend (or mixture), the liquid refrigerant composition and the refrigerant composition vapor in the evaporator (or in the condenser) may have different compositions. This may lead to inefficient systems and difficulties in servicing the equipment, thus a single component refrigerant composition is more desirable. An azeotrope or azeotrope-like composition will function essentially as a single component refrigerant composition in a chiller, such that the liquid composition and the vapor composition are essentially the same reducing any inefficiencies that might arise from the use of a non-azeotropic or non-azeotrope-like composition.

Chillers with cooling capacities above 700 kW generally employ flooded evaporators, where the refrigerant composition in the evaporator and the condenser surrounds a coil or tube bundle or other conduit for the heat transfer medium (i.e., the refrigerant composition is on the shell side). Flooded evaporators require larger charges of refrigerant composition, but permit closer approach temperatures and higher efficiencies. Chillers with capacities below 700 kW commonly employ evaporators with refrigerant composition flowing inside the tubes and heat transfer medium in the evaporator and the condenser surrounding the tubes, i.e., the heat transfer medium is on the shell side. Such chillers are called direct-expansion (DX) chillers. One embodiment of a water-cooled direct expansion chiller is illustrated in FIG. 2. In the chiller as illustrated in FIG. 2, first liquid heating medium, which is a warm liquid, such as warm water, enters evaporator 6′ at inlet 14. Mostly liquid refrigerant composition (with a small amount of refrigerant composition vapor) enters coil or tube bundle 9′ in evaporator 6′ at arrow 3′ and evaporates. As a result, first liquid heating medium is cooled in evaporator 6′, and a cooled first liquid heating medium exits evaporator 6′ at outlet 16, and is sent to a body to be cooled, such as a building. In this embodiment of FIG. 2, it is this cooled first liquid heating medium that cools the building or other body to be cooled. The refrigerant composition vapor exits evaporator 6′ at arrow 4′ and is sent to compressor 7′, where it is compressed and exits as high temperature, high pressure refrigerant composition vapor. This refrigerant composition vapor enters condenser 5′ through condenser coil or tube bundle 10′ at 1′. The refrigerant composition vapor is cooled by a second liquid heating medium, such as water, in condenser 5′ and becomes a liquid. The second liquid heating medium enters condenser 5′ through condenser heat transfer medium inlet 20. The second liquid heating medium extracts heat from the condensing refrigerant composition vapor, which becomes liquid refrigerant composition, and this warms the second liquid heating medium in condenser 5′. The second liquid heating medium exits through condenser heat transfer medium outlet 18. The condensed refrigerant composition liquid exits condenser 5′ through lower coil 10′ and flows through expansion device 12, which may be an orifice, capillary tube or expansion valve. Expansion device 12 reduces the pressure of the liquid refrigerant composition. A small amount of vapor, produced as a result of the expansion, enters evaporator 6′ with liquid refrigerant composition through coil 9′ and the cycle repeats.

Vapor-compression chillers may be identified by the type of compressor they employ. The present invention includes chillers utilizing centrifugal compressors as well as positive displacement compressors. In one embodiment, the compositions as disclosed herein are useful in chillers which utilizes a centrifugal compressor, herein referred to as a centrifugal chiller.

A centrifugal compressor uses rotating elements to accelerate the refrigerant composition radially, and typically includes an impeller and diffuser housed in a casing. Centrifugal compressors usually take working fluid in at an impeller eye, or central inlet of a circulating impeller, and accelerate it radially outward through passages. Some static pressure rise occurs in the impeller, but most of the pressure rise occurs in the diffuser section of the casing, where velocity is converted to static pressure. Each impeller-diffuser set is a stage of the compressor. Centrifugal compressors are built with from 1 to 12 or more stages, depending on the final pressure desired and the volume of refrigerant composition to be handled.

The pressure ratio, or compression ratio, of a compressor is the ratio of absolute discharge pressure to the absolute inlet pressure. Pressure delivered by a centrifugal compressor is practically constant over a relatively wide range of capacities. The pressure a centrifugal compressor can develop depends on the tip speed of the impeller. Tip speed is the speed of the impeller measured at its outermost tip and is related to the diameter of the impeller and its revolutions per minute. The capacity of the centrifugal compressor is determined by the size of the passages through the impeller. This makes the size of the compressor more dependent on the pressure required than the capacity.

In another embodiment, the compositions as disclosed herein are useful in positive displacement chillers, which utilize positive displacement compressors, either reciprocating, screw, or scroll compressors. A chiller which utilizes a screw compressor will be hereinafter referred to as a screw chiller.

Positive displacement compressors draw vapor into a chamber, and the chamber decreases in volume to compress the vapor. After being compressed, the vapor is forced from the chamber by further decreasing the volume of the chamber to zero or nearly zero.

Reciprocating compressors use pistons driven by a crankshaft. They may be either stationary or portable, may be single or multi-staged, and may be driven by electric motors or internal combustion engines. Small reciprocating compressors from 5 to 30 hp are seen in automotive applications and are typically for intermittent duty. Larger reciprocating compressors up to 100 hp are found in large industrial applications. Discharge pressures can range from low pressure to very high pressure (>5000 psi or 35 MPa).

Screw compressors use two meshed rotating positive-displacement helical screws to force the gas into a smaller space. Screw compressors are usually for continuous operation in commercial and industrial application and may be either stationary or portable. Their application can be from 5 hp (3.7 kW) to over 500 hp (375 kW) and from low pressure to very high pressure (>1200 psi or 8.3 MPa).

Scroll compressors are similar to screw compressors and include two interleaved spiral-shaped scrolls to compress the gas. The output is more pulsed than that of a rotary screw compressor.

For chillers which use scroll compressors or reciprocating compressors, capacities below 150 kW, brazed-plate heat exchangers are commonly used for evaporators instead of the shell-and-tube heat exchangers employed in larger chillers. Brazed-plate heat exchangers reduce system volume and refrigerant composition charge.

The compositions comprising HFC-245eb and optionally Z—HFO-1336mzz may be used in a chiller apparatus in combination with molecular sieves to aid in removal of moisture. Desiccants may comprise activated alumina, silica gel, or zeolite-based molecular sieves. In certain embodiments, the preferred molecular sieves have a pore size of approximately 3 Angstroms, 4 Angstroms, or 5 Angstroms. Representative molecular sieves include MOLSIV XH-7, XH-6, XH-9 and XH-11 (UOP LLC, Des Plaines, Ill.).

### Compositions

Of particular utility in the method for producing cooling and the chiller apparatus described herein are those embodiments wherein the refrigerant composition is a composition comprising HFC-245eb and optionally Z—HFO-1336mzz. Also of particular utility are those embodiments wherein the refrigerant composition is azeotropic or azeotrope-like. Because azeotropic and azeotrope-like compositions do not fractionate to any large degree, they function in a system with low glide in the evaporator of the chiller.

U.S. Provisional Patent Application Ser. No. 61/448,241, filed Mar. 2, 2011 (now WO2012/106565A1, published Aug. 9, 2012) discloses HFC-245eb and Z—HFO-1336mzz for azeotropic and azeotrope-like compositions.

Of note are compositions that provide less than 1° C. average temperature glide comprising less than or equal to about 57 weight percent Z—HFO-1336mzz and greater than or equal to about 43 weight percent HFC-245eb; or comprising greater than or equal to about 82 weight percent Z—HFO-1336mzz and less than or equal to about 18 weight percent HFC-245eb. Of particular note are compositions that provide less than 0.5° C. average temperature glide comprising less than or equal to about 35 weight percent Z—HFO-1336mzz and greater than or equal to about 65 weight percent HFC-245eb; or comprising greater than or equal to about 92 weight percent Z—HFO-1336mzz and less than or equal to about 8 weight percent HFC-245eb.

In another embodiment, non-flammable compositions are desirable for use in chillers. Of note are non-flammable compositions comprising at least 41 weight percent Z—HFO-1336mzz and no more than 59 weight percent HFC-245eb.

In one embodiment, is provided a composition comprising: (1) a refrigerant composition consisting essentially of HFC-245eb and Z—HFO-1336mzz; (2) a lubricant suitable for use in a chiller; wherein the Z—HFO-1336mzz in the refrigerant composition is at least about 41 weight percent.

Additionally, in another embodiment, chillers operated with Z—HFO-1336mzz/HFC-245eb blends containing about 71 weight percent or more Z—HFO-1336mzz or more will have vapor pressures below the threshold necessitating compliance with provisions of the ASME Boiler and Pressure Vessel Code. Such compositions are desirable for use in chillers. Of note are compositions where the refrigerant composition consists essentially of from about 71 to about 80 weight percent Z—HFO-1336mzz and from about 29 to 20 weight percent HFC-245eb.

Further, in another embodiment, low GWP compositions are desirable. Of note are compositions comprising at least 49.5 weight percent Z—HFO-1336mzz and HFC-245eb, which have GWP less than 150.

The compositions comprising HFC-245eb and Z—HFO-1336mzz may also comprise and/or be used in combination with at least one lubricant selected from the group consisting of polyalkylene glycols, polyol esters, polyvinylethers, mineral oils, alkylbenzenes, synthetic paraffins, synthetic naphthenes, and poly(alpha)olefins.

Useful lubricants include those suitable for use with chiller apparatus. Among these lubricants are those conventionally used in vapor compression refrigeration apparatus utilizing chlorofluorocarbon refrigerant compositions. In one embodiment, lubricants comprise those commonly known as “mineral oils” in the field of compression refrigeration lubrication. Mineral oils comprise paraffins (i.e., straight-chain and branched-carbon-chain, saturated hydrocarbons), naphthenes (i.e. cyclic paraffins) and aromatics (i.e. unsaturated, cyclic hydrocarbons containing one or more rings characterized by alternating double bonds). In one embodiment, lubricants comprise those commonly known as “synthetic oils” in the field of compression refrigeration lubrication. Synthetic oils comprise alkylaryls (i.e. linear and branched alkyl alkylbenzenes), synthetic paraffins and naphthenes, and poly(alphaolefins). Representative conventional lubricants are the commercially available BVM 100 N (paraffinic mineral oil sold by BVA Oils), naphthenic mineral oil commercially available from Crompton Co. under the trademarks Suniso® 3GS and Suniso® 5GS, naphthenic mineral oil commercially available from Pennzoil under the trademark Sontex® 372LT, naphthenic mineral oil commercially available from Calumet Lubricants under the trademark Calumet® RO-30, linear alkylbenzenes commercially available from Shrieve Chemicals under the trademarks Zerol® 75, Zerol® 150 and Zerol® 500, and HAB 22 (branched alkylbenzene sold by Nippon Oil).

Useful lubricants may also include those which have been designed for use with hydrofluorocarbon refrigerant compositions and are miscible with refrigerant compositions of the present invention under compression refrigeration and air-conditioning apparatus' operating conditions. Such lubricants include, but are not limited to, polyol esters (POEs) such as Castrol® 100 (Castrol, United Kingdom), polyalkylene glycols (PAGs) such as RL-488A from Dow (Dow Chemical, Midland, Mich.), polyvinyl ethers (PVEs), and polycarbonates (PCs).

Preferred lubricants are polyol esters.

Lubricants used with the refrigerant compositions disclosed herein are selected by considering a given compressor's requirements and the environment to which the lubricant will be exposed.

In one embodiment, the compositions as disclosed herein may further comprise an additive selected from the group consisting of compatibilizers, UV dyes, solubilizing agents, tracers, stabilizers, perfluoropolyethers (PFPE), and functionalized perfluoropolyethers.

In one embodiment, the compositions may be used with about 0.01 weight percent to about 5 weight percent of a stabilizer, free radical scavenger or antioxidant. Such other additives include but are not limited to, nitromethane, hindered phenols, hydroxylamines, thiols, phosphites, or lactones. Single additives or combinations may be used.

Optionally, in another embodiment, certain refrigeration or air-conditioning system additives may be added, as desired, to the in order to enhance performance and system stability. These additives are known in the field of refrigeration and air-conditioning, and include, but are not limited to, anti wear agents, extreme pressure lubricants, corrosion and oxidation inhibitors, metal surface deactivators, free radical scavengers, and foam control agents. In general, these additives may be present in the inventive compositions in small amounts relative to the overall composition. Typically concentrations of from less than about 0.1 weight percent to as much as about 3 weight percent of each additive are used. These additives are selected on the basis of the individual system requirements. These additives include members of the triaryl phosphate family of EP (extreme pressure) lubricity additives, such as butylated triphenyl phosphates (BTPP), or other alkylated triaryl phosphate esters, e.g. Syn-O-Ad 8478 from Akzo Chemicals, tricresyl phosphates and related compounds. Additionally, the metal dialkyl dithiophosphates (e.g., zinc dialkyl dithiophosphate (or ZDDP)), Lubrizol 1375 and other members of this family of chemicals may be used in compositions of the present invention. Other antiwear additives include natural product oils and asymmetrical polyhydroxyl lubrication additives, such as Synergol TMS (International Lubricants). Similarly, stabilizers such as antioxidants, free radical scavengers, and water scavengers may be employed. Compounds in this category can include, but are not limited to, butylated hydroxy toluene (BHT), epoxides, and mixtures thereof. Corrosion inhibitors include dodecyl succinic acid (DDSA), amine phosphate (AP), oleoyl sarcosine, imidazone derivatives and substituted sulfphonates.

### EXAMPLES

The concepts described herein will be further described in the following examples, which do not limit the scope of the invention described in the claims.

### Example 1

### Cooling Performance of “Neat” HFC-245eb

This example demonstrates the use of HFC-245eb as a refrigerant composition in a chiller. Comparison with performance for CFC-11 and HCFC-123 demonstrates use of HFC-245eb as a replacement for CFC-11 or HCFC-123 in chillers. In Table 1, Pevap is pressure of the evaporator; Pcond is pressure of the condenser; PR is pressure ratio (Pcond/Pevap); Utip is tip speed; COP is coefficient of performance (a measure of energy efficiency); and CAP is volumetric capacity. The performance for HFC-245eb, CFC-11 and HCFC-123 is determined for the following conditions:

| | | |
|---|---|---|
| Evaporator temperature | ° C. | 4.44 |
| Condenser temperature | ° C. | 37.78 |
| Superheat | ° C. | 0.00 |
| Subcool | ° C. | 0.00 |
| Compressor Efficiency | | 0.70 |

TABLE 1

| | | | | | HFC- |
|---|---|---|---|---|---|
| | | | | HFC- | 245eb |
| | | | | 245eb | vs |
| | | | | vs | HCFC- |
| | | HCFC- | HFC- | CFC-11 | 123 |
| | CFC-11 | 123 | 245eb | (%) | (%) |
| ODP | 1 | 0.02 | 0 | | |
| GWP | 4,750 | 77 | 286(#) | | |
| Pevap, MPa | 0.05 | 0.04 | 0.05 | | |
| Pcond, MPa | 0.16 | 0.14 | 0.18 | | |
| PR | 3.36 | 3.61 | 3.85 | | |
| Compressor | 21.147 | 19.733 | 23.639 | 11.78 | 19.79 |
| Work- | | | | | |
| Isentropic, | | | | | |
| kJ/kg | | | | | |
| Utip, m/sec | 196.08 | 189.42 | 207.32 | 5.73 | 9.45 |
| COP | 5.268 | 5.103 | 5.265 | −0.06 | 3.17 |
| CAP, kJ/m³ | 464.01 | 386.51 | 484.81 | 4.48 | 25.43 |

(#)Rajakumar, B., R. W. Portmann, et al. (2006). “Rate Coefficients for the Reactions of OH with CF₃CH₂CH₃ (HFC-263fb), CF₃CHFCH₂F (HFC-245eb), and CHF₂CHFCHF₂ (HFC-245ea) between 238 and 375 K†.” The Journal of Physical Chemistry A, 110(21): 6724-6731.

Neat HFC-245eb has attractive environmental properties (a relatively low GWP and zero ODP). It also exhibits attractive chiller performance (a high COP for cooling and high volumetric cooling capacity). Chiller COP with HFC-245eb matches the COP with CFC-11 and exceeds the COP with HCFC-123 by 3.17%. Chiller volumetric cooling capacity with HFC-245eb exceeds the volumetric capacity with CFC-11 by 4.48% and with HCFC-123 by 25.43%. The impeller tip speed with HFC-245eb required to meet a cooling duty will be only slightly higher than with CFC-11 (by 5.73%) or HCFC-123 (by 9.45%). HFC-245eb would be a suitable near drop-in replacement of CFC-11 in centrifugal chillers and would enable chillers with substantially better performance than HCFC-123. HFC-245eb could be used as a replacement for HCFC-123 in existing chillers if vessels compliant with the ASME Boiler and Pressure Vessel Code were used and suitable flammability mitigation measures were put in place.

### Example 2

### Cooling Performance for a Non-Flammable Blend of Z—HFO-1336mzz and HFC-245eb

This example demonstrates chiller performance with a non-flammable blend containing 41 weight percent Z—HFO-1336mzz and 59 weight percent HFC-245eb. In Table 2, Pevap is pressure of the evaporator; Pcond is pressure of the condenser; PR is pressure ratio (Pcond/Pevap); Utip is tip speed; COP is coefficient of performance (a measure of energy efficiency); and CAP is volumetric capacity. The performance for a blend of HFC-245eb and Z—HFO-1336mzz and for CFC-11 and HCFC-123 is determined for the following conditions:

| Evaporator temperature | 4.44° C. |
|---|---|
| Condenser temperature | 37.78° C. |
| Superheat | 0.00° C. |
| Subcool | 0.00° C. |
| Compressor Efficiency | 0.70 |

TABLE 2

| | | | | HFC- | HFC- |
|---|---|---|---|---|---|
| | | | HFC- | 245eb/ | 245eb/ |
| | | | 245eb/ | Z-HFO- | Z-HFO- |
| | | | Z-HFO- | 1336mzz | 1336mzz |
| | | | 1336mzz | (59/41) | (59/41) vs |
| | | HCFC- | (59/41 | vs | HCFC- |
| | CFC-11 | 123 | wt %) | CFC-11 | 123 |
| ODP | 1 | 0.02 | 0 | | |
| GWP | 4,750 | 77 | 173 | | |
| Pevap, MPa | 0.05 | 0.04 | 0.04 | | |
| Pcond, MPa | 0.16 | 0.14 | 0.16 | | |
| PR | 3.36 | 3.61 | 3.82 | | |
| Evap | 0.00 | 0.00 | 0.52 | | |
| Glide, ° C. | | | | | |
| Cond | 0.00 | 0.00 | 0.77 | | |
| Glide, ° C. | | | | | |
| Average | 0.00 | 0.00 | 0.65 | | |
| Glide, ° C. | | | | | |
| Compressor | 21.15 | 19.73 | 21.64 | 2.32 | 9.65 |
| Work, | | | | | |
| Isentropic, | | | | | |
| kJ/kg | | | | | |
| Utip, m/sec | 196.08 | 189.42 | 198.34 | 1.15 | 4.71 |
| COP | 5.27 | 5.10 | 5.17 | −1.90 | 1.27 |
| CAP, kJ/m³ | 464.01 | 386.51 | 429.23 | −7.49 | 11.05 |

A composition containing of about 41 weight percent Z—HFO-1336mzz and 59 weight percent HFC-245eb is non-flammable, nearly azeotropic with temperature glide at chiller conditions lower than 1° C., has a low GWP of 173 and zero ODP. Moreover, it would exhibit attractive chiller performance (high COP for cooling and high volumetric cooling capacity) as shown in Table 2. Chiller COP and capacity with the above blend would nearly match CFC-11 and would exceed HCFC-123. The impeller tip speed with the above blend required to meet a cooling duty will be only slightly higher than with CFC-11 (by 1.15%) or HCFC-123 (by 4.71%). The above blend makes a suitable near drop-in replacement for CFC-11 in chillers and would enable chillers with substantially better performance than HCFC-123. It could be a replacement for HCFC-123 in existing chillers if vessels compliant with the ASME Boiler and Pressure Vessel Code were used.

### Example 3

### Cooling Performance for a Lower Vapor Pressure Blend of Z—HFO-1336mzz and HFC-245eb

The vapor pressure of Z—HFO-1336mzz/HFC-245eb blends decreases as the Z—HFO-1336mzz content of the blend increases. Chillers operated with Z—HFO-1336mzz/HFC-245eb blends containing about 71 weight percent or more Z—HFO-1336mzz will have vapor pressures below the threshold necessitating compliance with provisions of the ASME Boiler and Pressure Vessel Code. In Table 3, Pevap is pressure of the evaporator; Pcond is pressure of the condenser; PR is pressure ratio (Pcond/Pevap); Utip is tip speed; COP is coefficient of performance (a measure of energy efficiency); and CAP is volumetric capacity. The performance for a blend of HFC-245eb and Z—HFO-1336mzz and for CFC-11 and HCFC-123 is determined for the following conditions:

| Evaporator temperature | 4.44° C. |
|---|---|
| Condenser temperature | 37.78° C. |
| Superheat | 0.00° C. |
| Subcool | 0.00° C. |
| Compressor Efficiency | 0.70 |

TABLE 3

| | | | | HFC- | HFC- |
|---|---|---|---|---|---|
| | | | | 245eb/ | 245eb/ |
| | | | HFC- | Z-HFO- | Z-HFO- |
| | | | 245eb/ | 1336mzz | 1336mzz |
| | | | Z-HFO- | (59/41) | (59/41) vs |
| | | | 1336mzz | vs | HCFC- |
| | | HCFC- | (29/71) | CFC-11 | 123 |
| | CFC-11 | 123 | wt %) | (%) | (%) |
| ODP | 1 | 0.02 | 0 | | |
| GWP | 4,750 | 77 | 90 | | |
| Pevap, | 0.05 | 0.04 | 0.04 | −23.19 | −7.26 |
| MPa | | | | | |
| Pcond, | 0.16 | 0.14 | 0.14 | −12.35 | −1.45 |
| MPa | | | | | |
| PR | 3.36 | 3.61 | 3.83 | 14.12 | 6.26 |
| Evap | 0.00 | 0.00 | 0.92 | | |
| Glide, ° C. | | | | | |
| Cond | 0.00 | 0.00 | 1.33 | | |
| Glide, ° C. | | | | | |
| Average | 0.00 | 0.00 | 1.13 | | |
| Glide, ° C. | | | | | |
| Compres- | 21.15 | 19.73 | 20.41 | −3.48 | 3.44 |
| sor Work, | | | | | |
| Isentropic, | | | | | |
| kJ/kg | | | | | |
| Utip, | 196.08 | 189.42 | 192.65 | −1.75 | 1.71 |
| m/sec | | | | | |
| COP | 5.268 | 5.103 | 5.082 | −3.53 | −0.41 |
| CAP, | 464.01 | 386.51 | 371.77 | −19.88 | −3.81 |
| kJ/m³ | | | | | |

Table 3 shows that a Z—HFO-1336mzz/HFC-245eb blend containing 71 weight percent Z—HFO-1336mzz could be used to replace HCFC-123 in chillers. It could also be used to replace CFC-11 in chillers when a modest cooling capacity loss would be acceptable. In some applications, some loss of cooling capacity may be acceptable (e.g., when the nominal chiller cooling rate is higher than that actually needed) or may be compensated by supplying additional cooling by other means (e.g., additional chilled water from other chillers) or by reducing the cooling load.

What is claimed is:

1. A method for producing cooling in a chiller having an evaporator wherein a refrigerant composition is evaporated to cool a heat transfer medium and the cooled heat transfer medium is transported out of the evaporator to a body to be cooled, wherein the method comprising: evaporating a refrigerant composition comprising HFC-245eb and optionally Z—HFO-1336mzz in the evaporator.

2. The method of claim 1, wherein said step of evaporating the composition produces a vapor composition, and further comprising the step of compressing the vapor composition in a compressor, wherein the compressor is a centrifugal compressor.

3. The method of claim 1 wherein the evaporator is suitable for use with HCFC-123 and wherein the refrigerant composition consists essentially of from about 1 weight percent to about 59 weight percent HFC-245eb and from about 41 weight percent to about 99 weight percent Z—HFO-1336mzz.

4. The method of claim 1 wherein the chiller is suitable for use with HCFC-123 and wherein the refrigerant composition consists essentially of from about 1 weight percent to about 29 weight percent HFC-245eb and from about 71 weight percent to about 99 weight percent Z—HFO-1336mzz.

5. The method of claim 1 wherein the chiller is suitable for use with CFC-11 and wherein the refrigerant composition consists essentially of from about 1 weight percent to about 59 weight percent HFC-245eb and from about 41 weight percent to about 99 weight percent Z—HFO-1336mzz.

6. The method of claim 1 wherein the refrigerant composition consists essentially of HFC-245eb.

7. The method of claim 1 wherein the refrigerant composition consists essentially of HFC-245eb and Z—HFO-1336mzz; and wherein the Z—HFO-1336mzz in the refrigerant composition is at least about 1 weight percent.

8. The method of claim 7 wherein the refrigerant composition consists essentially of from about 99 weight percent to about 43 weight percent HFC-245eb and from about 1 weight percent to about 57 weight percent Z—HFO-1336mzz.

9. The method of claim 7 wherein the refrigerant composition consists essentially of from about 1 weight percent to about 18 weight percent HFC-245eb and from about 82 weight percent to about 99 weight percent Z—HFO-1336mzz.

10. A composition comprising: (1) a refrigerant composition consisting essentially of HFC-245eb and Z—HFO-1336mzz; (2) a lubricant suitable for use in a chiller; wherein the Z—HFO-1336mzz in the refrigerant composition is at least about 41 weight percent.

11. The composition of claim 10 wherein the refrigerant composition consists essentially of from about 1 weight percent to about 59 weight percent HFC-245eb and from about 41 weight percent to about 99 weight percent Z—HFO-1336mzz.

12. A chiller apparatus containing a refrigerant composition, characterized by:
said refrigerant composition comprising HFC-245eb and optionally Z—HFO-1336mzz.

13. The chiller apparatus of claim 12 comprising (a) an evaporator through which a refrigerant flows and is evaporated; (b) a compressor in fluid communication with the evaporator that compresses the evaporated refrigerant to a higher pressure; (c) a condenser in fluid communication with the compressor through which the high pressure refrigerant vapor flows and is condensed; and (d) a pressure reduction device in fluid communication with the condenser wherein the pressure of the condensed refrigerant is reduced and said pressure reduction device further being in fluid communication with the evaporator such that the refrigerant then repeats flow through components (a), (b), (c) and (d) in a repeating cycle.
